(19) United States
(12) Patent Application Publication
(10) Pub. No.: US 2023/0074311 A1
(43) Pub. Date: Mar. 9, 2023

(54) COMPOSITION FOR CANCER DIAGNOSIS

(71) Applicant: BERTIS INC, Seongnam-si, Gyeonggi-do (KR)

(72) Inventors: Sung Soo KIM, Yongin-si (KR); Seung Man HAN, Seoul (KR)

(73) Assignee: BERTIS INC, Seongnam-si, Gyeonggi-do (KR)

(21) Appl. No.: 17/793,158

(22) PCT Filed: Jan. 15, 2020
(86) PCT No.: PCT/KR2020/000749

§ 371 Date: Jul. 15, 2022

(51) Int. Cl. G01N 33/574

(52) U.S. Cl. CPC G01N 33/57415; G01N 2333/912

(57) ABSTRACT

The present invention relates to a composition capable of cancer diagnosis, a diagnostic kit comprising same, and a method for providing information for cancer diagnosis using the composition. When a biomarker of the present invention is used, it is possible to accurately and conveniently diagnose cancer, particularly breast cancer, in an early stage, and furthermore, it is possible to diagnose the stage of cancer and predict therapeutic responsiveness or post-treatment prognosis.

### TECHNICAL FIELD

The present invention relates to a composition capable of diagnosing cancer, a diagnostic kit comprising the same, and a method of providing information for cancer diagnosis using the composition.

### BACKGROUND ART

Breast cancer is the second most common cancer after lung cancer in the world, and is a dangerous cancer that ranks fifth in mortality rate. In particular, in recent years, in women undergoing physiologically vigorous physical changes, such as low fertility, short lactation, early menarche, and late menopause, incidence of breast cancer has increased rapidly due to the increased sensitivity of mammary gland tissue by a rapid increase in the number of times stimulated by female hormones, the westernization of eating habits, contamination of the living environment, etc. In the case of breast cancer, once cancer cells invade surrounding tissues or start to metastasize to lymph nodes, it is difficult to cure breast cancer. Thus, it can be said that early diagnosis of breast cancer is more important than other cancers.

In order to reduce the mortality rate caused by breast cancer, it is important to (1) diagnose breast cancer early, and (2) predict the prognosis after treatment by primary surgery and perform appropriate adjuvant therapy. Currently, for the diagnosis of breast cancer, in addition to self-diagnosis by primary palpation, mammography, ultrasonography, etc. are used as medical examination methods for preventive purposes. These methods are also most widely used to diagnose early breast cancer. However, mammography has a disadvantage in that the diagnosis rate of breast cancer is low in the case of dense breasts commonly found in Korean women because the breasts have a lot of fibers, and the diagnosis rate is also low, particularly in the case of young women with well-developed mammary glands. In addition, due to the use of X-rays, the likelihood of developing breast cancer during the diagnosis process cannot be excluded. Hence, ultrasonography is used as an alternative, but even with this method, it is difficult to distinguish between malignant tumors (cancer) and benign tumors (non-cancer). In actual clinical practice, if there are abnormal findings, fine needle aspiration cytology, magnetic resonance imaging, etc. are additionally used to increase the diagnosis rate.

However, even with these methods, it is merely possible to morphologically distinguish between normal tissue and abnormal tissue, and it is difficult to distinguish between malignant tumors (cancer) and benign tumors (non-cancer). For confirmation of breast cancer, a more precise biopsy is performed.

For these reasons, methods of diagnosing breast cancer by a molecular genetic method have been much more developed than other cancers. In biopsy, the tissue is cut to confirm the lesion, and primary surgery for excision is performed. To determine how to conduct subsequent adjuvant therapy, a method is used, which determines the presence or absence of estrogen receptor (ER) and the number of erythroblastic oncogene B-2 (ERBB2, also known as HER2/neu) gene, which is a breast cancer-specific tumor marker, by in situ hybridization. If the detected cancer tissue has an estrogen receptor, treatment is performed using an estrogen analogue such as tamoxifen, and if the erythroblastic oncogene B-2 (ERBB2) gene is overexpressed, the erythroblastic oncogene B-2 (ERBB2) monoclonal antibody trastuzumab commercially available under the trade name Herceptin is used for the treatment of breast cancer. Amplification and overexpression of the erythroblastic oncogene B-2 (ERBB2) gene, which is a breast cancer-specific diagnostic marker most useful for diagnosis and treatment of breast cancer, is found in 20 to 35% of invasive breast cancer. Thus, the erythroblastic oncogene B-2 (ERBB2) test together with the estrogen receptor test plays a crucial role in the treatment of breast cancer patients. Accordingly, various attempts have been made to detect the expression level of erythroblastic oncogene B-2 (ERBB2) more quickly and reliably.

### INVENTION

### Technical Problem

An object of the present invention is to provide a composition capable of accurately and conveniently diagnosing cancer, particularly breast cancer.

Another object of the present invention is to provide a kit capable of accurately and conveniently diagnosing cancer, particularly breast cancer.

Still another object of the present invention is to provide a method for providing information for diagnosing cancer, particularly breast cancer.

Yet another object of the present invention is to provide a method for predicting the therapeutic responsiveness of cancer, particularly breast cancer.

Still yet another object of the present invention is to provide a method for predicting the prognosis of cancer, particularly breast cancer.

A further object of the present invention is to provide a method for predicting the stage of cancer, particularly breast cancer.

Another further object of the present invention is to provide a method for predicting the likelihood of recurrence of cancer, particularly breast cancer.

Still another further object of the present invention is to provide a method for screening a drug for treating cancer, particularly breast cancer.

However, objects to be achieved by the present invention are not limited to the objects mentioned above, and other objects not mentioned herein will be clearly understood by those of ordinary skill in the art from the following description.

### Technical Solution

According to one embodiment of the present invention, the present invention is directed to a cancer diagnostic marker comprising a polypeptide represented by any one of SEQ ID NOs: 2 and 3.

In the cancer diagnostic marker according to the present invention, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma, or pituitary adenoma.

According to another embodiment of the present invention, the present invention is directed to a cancer diagnostic composition containing an agent for measuring the expression level of either a polypeptide represented by any one of SEQ ID NOs: 2 and 3, or a gene encoding the polypeptide.

In the cancer diagnostic composition according to the present invention, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma, or pituitary adenoma.

In the cancer diagnostic composition according to the present invention, the agent for measuring the expression level of the polypeptide represented by any one of SEQ ID NOs: 2 and 3 may comprise at least one selected from the group consisting of an antibody, an oligopeptide, a ligand, a peptide nucleic acid (PNA) and an aptamer, which bind specifically to the polypeptide.

In the cancer diagnostic composition according to the present invention, the agent for measuring the expression level of the gene encoding the polypeptide represented by any one of SEQ ID NOs: 2 and 3 may comprise at least one selected from the group consisting of a primer, a probe and an antisense nucleotide, which bind specifically to the gene encoding the polypeptide.

According to still another embodiment of the present invention, the present invention is directed to a cancer diagnostic kit comprising the cancer diagnostic composition.

In the present invention, the kit may be an RT-PCR kit, a DNA chip kit, an ELISA kit, a protein chip kit, a rapid kit or a multiple-reaction monitoring (MRM) kit.

According to yet another embodiment of the present invention, the present invention is directed to a method for providing information for cancer diagnosis, the method comprising a step of measuring the expression level of either a polypeptide represented by any one of SEQ ID NOs: 2 and 3, or a gene encoding the polypeptide, in a biological sample isolated from a subject of interest.

In the method for providing information for cancer diagnosis according to the present invention, the biological sample may be whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

In the present invention, the agent for measuring the expression level of the polypeptide represented by any one of SEQ ID NOs: 2 and 3 may comprise at least one selected from the group consisting of an antibody, an oligopeptide, a ligand, a peptide nucleic acid (PNA) and an aptamer, which bind specifically to the polypeptide represented by any one of SEQ ID NOs: 2 and 3.

In the method for providing information for cancer diagnosis according to the present invention, the measurement of the expression level of the polypeptide represented by any one of SEQ ID NOs: 2 and 3 may be performed by protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoeletrophoresis, immunohistochemical staining, complement fixation assay, two-dimensional electrophoresis assay, liquid chromatography-mass spectrometry (LC-MS), liquid chromatography-mass spectrometry/mass spectrometry (LC-MS/MS), Western blotting, or ELISA (enzyme-linked immunosorbent assay).

In the method for providing information for cancer diagnosis according to the present invention, the measurement of the expression level of the polypeptide represented by any one of SEQ ID NOs: 2 and 3 may be performed by a multiple-reaction monitoring (MRM) method.

Regarding the polypeptide represented by any one of SEQ ID NOs: 2 and 3 according to the present invention, a pair of mother and daughter ions of the polypeptide represented by SEQ ID NO: 2 as a target peptide may have mass-to-charge ratios of 370.724 m/z and 612.383, 513.314, 416.262, 303.178, 306.695, 257.161, 208.634 and 152.092 m/z, respectively, and a pair of mother and daughter ions of the polypeptide represented by SEQ ID NO: 3 as a target peptide may have mass-to-charge ratios of 402.247 m/z and 603.371, 502.324, 373.281, 260.197, 358.731, 302.189, 251.665, 187.144 and 130.602 m/z, respectively.

In the method for providing information for cancer diagnosis according to the present invention, an internal standard substance that is used in the multiple-reaction monitoring method may be either a synthetic peptide obtained by substituting certain amino acids of the target peptide with an isotope, or E. coli beta-galactosidase.

In the present invention, the target peptide of the E. coli beta-galactosidase may consist of a polypeptide represented by SEQ ID NO: 8, and mother and daughter ions thereof may have mass-to-charge ratios of 542.3 m/z and 636.3 m/z, respectively.

In the present invention, the agent for measuring the expression level of the gene encoding the polypeptide represented by any one of SEQ ID NOs: 2 and 3 may comprise at least one selected from the group consisting of a primer, a probe and an antisense nucleotide, which bind specifically to the gene encoding the polypeptide.

In the present invention, the measurement of the expression level of the gene encoding the polypeptide represented by any one of SEQ ID NOs: 2 and 3 may be performed by reverse transcription-polymerase chain reaction (RT-PCR), competitive RT-PCR, real-time RT-PCR, RNase protection assay (RPA), Northern blotting, or DNA chip assay.

In the present invention, if the measured expression level of either the polypeptide represented by any one of SEQ ID NOs: 2 and 3, or the gene encoding the polypeptide, in the biological sample isolated from the subject of interest, increases compared to a normal control, it may be predicted that the subject has a high likelihood of developing the cancer.

In the present invention, the method for providing information may be a method of predicting the prognosis of the subject of interest after surgical operation.

In the present invention, the method for providing information may be a method of diagnosing the stage of cancer in the subject of interest.

In the present invention, the method for providing information may be a method of predicting the likelihood of recurrence of cancer in the subject of interest.

In the present invention, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

According to another embodiment of the present invention, the present invention is directed to a method for screening a drug for preventing or treating cancer, the method comprising steps of: (a) treating either a sample isolated from a cancer subject or a cancer disease animal model with a candidate drug; and (b) measuring the expression level of either a polypeptide represented by any one of SEQ ID NOs: 2 and 3, or a gene encoding the polypeptide, in the sample or cancer disease animal model treated with the candidate drug.

In the present invention, the sample may be cells or tissue isolated from the cancer subject.

In the present invention, the method may further comprise step (c) of determining that the candidate drug is the drug for preventing or treating cancer, when the expression level of the polypeptide represented by any one of SEQ ID NOs: 2 and 3, or the gene encoding the polypeptide, measured in step (b), decreases or increases compared to that before treatment with the candidate drug.

In the present invention, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

### Advantageous Effects

When the biomarker of the present invention, which comprises the polypeptide represented by any one of SEQ ID NOs: 2 and 3 is used, it is possible to accurately and conveniently diagnose cancer, particularly breast cancer, in an early stage, and furthermore, it is possible to diagnose the stage of cancer and predict therapeutic responsiveness or post-treatment prognosis.

### BRIEF DESCRIPTION OF DRAWINGS

FIG. 1 shows a receiver operating characteristic (ROC) graph obtained based on the results of quantifying the biomarker erythroblastic oncogene B-2 (ERBB2) using a polypeptide represented by SEQ ID NO: 2 as a target peptide between breast cancer patients and a non-patient control group in Example 1 of the present invention.

FIG. 2 shows a receiver operating characteristic (ROC) graph obtained based on the results of quantifying the biomarker erythroblastic oncogene B-2 (ERBB2) using a polypeptide represented by SEQ ID NO: 3 as a target peptide between breast cancer patients and a non-patient control group in Example 1 of the present invention.

FIG. 3 shows a receiver operating characteristic (ROC) graph obtained based on the results of quantifying the biomarker erythroblastic oncogene B-2 (ERBB2) using a combination of polypeptides represented by SEQ ID NOs: 2 and 3 as a target peptide between breast cancer patients and a non-patient control group in Example 1 of the present invention.

FIG. 4 shows a receiver operating characteristic (ROC) graph obtained based on the results of quantifying the biomarker erythroblastic oncogene B-2 (ERBB2) using a polypeptide represented by SEQ ID NO: 4 as a target peptide between breast cancer patients and a non-patient control group in Comparative Example 1 of the present invention.

FIG. 5 shows a receiver operating characteristic (ROC) graph obtained based on the results of quantifying the biomarker erythroblastic oncogene B-2 (ERBB2) using a polypeptide represented by SEQ ID NO: 5 as a target peptide between breast cancer patients and a non-patient control group in Comparative Example 1 of the present invention.

FIG. 6 shows a receiver operating characteristic (ROC) graph obtained based on the results of quantifying the biomarker erythroblastic oncogene B-2 (ERBB2) using a polypeptide represented by SEQ ID NO: 6 as a target peptide between breast cancer patients and a non-patient control group in Comparative Example 1 of the present invention.

FIG. 7 shows a receiver operating characteristic (ROC) graph obtained based on the results of quantifying the biomarker erythroblastic oncogene B-2 (ERBB2) using a polypeptide represented by SEQ ID NO: 7 as a target peptide between breast cancer patients and a non-patient control group in Comparative Example 1 of the present invention.

### BEST MODE

One embodiment of the present invention is directed to a cancer diagnostic marker comprising a polypeptide represented by any one of SEQ ID NOs: 2 and 3.

### MODE FOR INVENTION

According to one embodiment of the present invention, the present invention is directed to a cancer diagnostic marker comprising a polypeptide represented by any one of SEQ ID NOs: 2 and 3.

In the present invention, the cancer diagnostic marker may be erythroblastic oncogene B-2 (ERBB2).

In the present invention, the erythroblastic oncogene B-2 (ERBB2) may consist of the amino acid sequence shown in SEQ ID NO: 1, without being limited thereto.

In the present invention, the “erythroblastic oncogene B-2 (ERBB2)” belongs to the epidermal growth factor receptor (EGFR) family and is a tyrosine kinase receptor which is involved in cell growth and differentiation signaling and displayed on the cell membrane surface. ERBB2 is also named HER2/neu, and is known to be involved in the formation of many tumors, including the occurrence of breast cancer, and thus has been much studied as a target for tumor treatment. In the present invention, the erythroblastic oncogene B-2 (ERBB2) protein may consist of the amino acid sequence shown in SEQ ID NO: 1, without being limited thereto.

In the present invention, the cancer diagnostic marker preferably comprises both the polypeptide represented by SEQ ID NO: 2 and the polypeptide represented by SEQ ID NO: 3.

In the present invention, the term “cancer” as a disease to be diagnosed represents or refers to a physiological condition characterized by atypical and uncontrolled cell growth in mammals. In the present invention, the cancer to be diagnosed may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

According to another embodiment of the present invention, the present invention is directed to a cancer diagnostic composition containing an agent for measuring the expression level of either a polypeptide represented by any one of SEQ ID NOs: 2 and 3, or a gene encoding the polypeptide.

In the present invention, the agent for measuring the expression level of the polypeptide may be an agent for measuring the expression level of erythroblastic oncogene B-2 (ERBB2).

In the present invention, the agent for measuring the expression level preferably comprises both an agent for measuring the expression level of the polypeptide represented by SEQ ID NO: 2, and an agent for measuring the expression level of the polypeptide represented by SEQ ID NO: 3.

In the present invention, the agent for measuring the expression level preferably comprises both an agent for measuring the expression level of a gene encoding the polypeptide represented by SEQ ID NO: 2, and an agent for measuring the expression level of a gene encoding the polypeptide represented by SEQ ID NO: 3.

In the present invention, the cancer to be diagnosed may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

In the present invention, the “diagnosis” or “diagnosing” includes: determining the susceptibility of a subject to a specific disease or disorder; determining whether or not a subject currently has a particular disease or disorder; determining the prognosis of a subject with a specific disease or disorder (e.g., identification of pre-metastatic or metastatic cancer conditions, determination of cancer stages, or determination of responsiveness of cancer to therapy); or therametrics (e.g., monitoring states of a subject to provide information about treatment effects). With regard to the purposes of the present invention, the diagnosis or diagnosing refers to determining whether or not the above-described cancer has developed or the likelihood (risk) of developing the cancer.

In the present invention, the agent for measuring the expression level of the polypeptide represented by any one of SEQ ID NOs: 2 and 3 is not particularly limited, but may comprise, for example, at least one selected from the group consisting of an antibody, an oligopeptide, a ligand, a peptide nucleic acid (PNA) and an aptamer, which bind specifically to the polypeptide.

In the present invention, the “antibody” refers to a substance that binds specifically to an antigen, causing an antigen-antibody reaction. With regard to the purposes of the present invention, the antibody refers to an antibody that binds specifically to the polypeptide represented by SEQ ID NO: 2 or 3.

The antibodies of the present invention include all polyclonal antibodies, monoclonal antibodies, and recombinant antibodies. The antibody may be easily produced using techniques well known in the art. For example, the polyclonal antibody may be produced by a method well known in the art, which comprises a process of injecting the protein antigen into an animal, collecting blood from the animal, and isolating serum containing the antibody. This polyclonal antibody may be produced from any animal species such as goats, rabbits, sheep, monkeys, horses, pigs, cattle, or dogs. In addition, the monoclonal antibody may be produced using a hybridoma method (see Kohler and Milstein (1976) European Journal of Immunology 6:511-519) well known in the art, or phage antibody library technology (see Clackson et al, Nature, 352:624-628, 1991; Marks et al, J. Mol. Biol., 222:58, 1-597, 1991). The antibody produced by the above method may be isolated and purified using methods such as gel electrophoresis, dialysis, salt precipitation, ion exchange chromatography, and affinity chromatography. In addition, the antibodies of the present invention include functional fragments of antibody molecules as well as complete forms having two full-length light chains and two full-length heavy chains. The expression “functional fragments of antibody molecules” refers to fragments retaining at least an antigen-binding function, and examples of the functional fragments include Fab, F(ab∝), F(ab∝)2, and Fv.

In the present invention, “peptide nucleic acid (PNA)” refers to an artificially synthesized polymer similar to DNA or RNA, and was first introduced by professors Nielsen, Egholm, Berg and Buchardt (at the University of Copenhagen, Denmark) in 1991. DNA has a phosphate-ribose backbone, whereas PNA has a backbone composed of repeating units of N-(2-aminoethyl)-glycine linked by peptide bonds. Thanks to this structure, PNA has a significantly increased binding affinity for DNA or RNA and a significantly increased stability, and thus is used in molecular biology, diagnostic analysis, and antisense therapy. PNA is disclosed in detail in Nielsen P E, Egholm M, Berg R H, Buchardt O (December 1991). “Sequence-selective recognition of DNA by strand displacement with a thymine-substituted polyamide”. Science 254 (5037): 1497-1500.

In the present invention, the “aptamer” is an oligonucleic acid or peptide molecule, and general contents of the aptamer are disclosed in detail in Bock L C et al., Nature 355(6360):5646(1992); Hoppe-Seyler F, Butz K “Peptide aptamers: powerful new tools for molecular medicine”. J Mol Med. 78(8):42630(2000); Cohen B A, Colas P, Brent R. “An artificial cell-cycle inhibitor isolated from a combinatorial library”. Proc Natl Acad Sci USA. 95(24): 142727(1998).

In the present invention, the agent for measuring the expression level of the gene encoding the polypeptide represented by any one of SEQ ID NOs: 2 and 3 may comprise at least one selected from the group consisting of a primer, a probe and an antisense nucleotide, which bind specifically to the gene encoding the polypeptide.

In the present invention, the “primer” is a fragment that recognizes a target gene sequence, and includes a pair of forward and reverse primers. Preferably, the primer is a primer pair that provides analysis results with specificity and sensitivity. Because the nucleotide sequence of the primer does not match a non-targeted sequence in a sample, the primer can show high specificity when it amplifies only a target gene sequence containing a complementary primer binding site without causing non-specific amplification.

In the present invention, the “probe” refers to a substance which is capable of binding specifically to the target substance to be detected in a sample and may specifically identify the presence of the target substance in the sample through the binding. The kind of the probe is not specifically limited, as long as it is a substance that is generally used in the art. Preferably, the probe may be peptide nucleic acid (PNA), locked nucleic acid (LNA), a peptide, a polypeptide, a protein, RNA or DNA. Most preferably, the probe is PNA. More specifically, the probe may be a biomaterial derived from an organism, an analogue thereof, or a material produced ex vivo, and examples thereof include enzymes, proteins, antibodies, microorganisms, animal/plant cells and organs, neural cells, DNA, and RNA. Examples of the DNA include cDNA, genomic DNA, and oligonucleotides, examples of the RNA include genomic RNA, mRNA, and oligonucleotides, and examples of the protein include antibodies, antigens, enzymes, and peptides.

In the present invention, the “locked nucleic acid (LNA)” refers to a nucleic acid analog containing a 2′-O or 4′-C methylene bridge [J Weiler, J Hunziker and J Hall Gene Therapy (2006) 13, 496.502]. LNA nucleosides include common nucleic acid bases of DNA and RNA, and can form base pairs according to the Watson-Crick base pairing rule. However, due to ‘locking’ of the molecule attributable to the methylene bridge, the LNA fails to form an ideal shape in the Watson-Crick bond. When the LNA is incorporated in a DNA or RNA oligonucleotide, it can more rapidly pair with a complementary nucleotide chain, thus increasing the stability of the double strand. In the present invention, the “antisense” refers to an oligomer having a sequence of nucleotide bases and a subunit-to-subunit backbone that allows the antisense oligomer to hybridize to a target sequence in an RNA by Watson-Crick base pairing, to form an RNA:oligomer heteroduplex within the target sequence, typically with an mRNA. The oligomer may have exact sequence complementarity to the target sequence or near complementarity.

The amino acid sequence of the polypeptide according to the present invention is represented by SEQ ID NO: 2 or 3. Thus, based on this amino acid sequence information, those skilled in the art can easily design a primer, a probe or an antisense nucleotide, which specifically binds to the gene encoding the polypeptide.

According to still another embodiment of the present invention, the present invention is directed to a cancer diagnostic kit comprising the cancer diagnostic composition according to the present invention.

In the present invention, it is possible to diagnose the development, likelihood of development, responsiveness to therapy, prognosis, stage, likelihood of recurrence, etc. of a cancer disease by using the diagnostic kit. In the present invention, the cancer to be diagnosed may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

In the present invention, the kit may be, but is not limited to, an RT-PCR kit, a DNA chip kit, an ELISA kit, a protein chip kit, a rapid kit or a multiple-reaction monitoring (MRM) kit.

The cancer diagnostic kit of cancer according to the present invention may further include one or more other component compositions, solutions or devices suitable for analysis methods.

For example, the cancer diagnostic kit according to the present invention may further comprise essential elements necessary for performing reverse transcription polymerase reaction. The reverse transcription polymerase reaction kit comprises a pair of primers specific to a gene encoding a marker protein. Each primer is a nucleotide having a sequence specific to the nucleic acid sequence of the gene, and may have a length of about 7 bp to 50 bp, more preferably about 10 bp to 30 bp. In addition, the kit may comprise primers specific to the nucleic acid sequence of a control gene. In addition, the reverse transcription polymerase reaction kit may comprise a test tube or other suitable container, buffers (having various pHs and magnesium concentrations), deoxynucleotides (dNTPs), enzymes such as Taq-polymerase and reverse transcriptase, DNAse and RNAse inhibitors, DEPC-water, sterile water, and the like.

In addition, the diagnostic kit of the present invention may comprise essential elements necessary for performing DNA chip assay. The DNA chip kit may comprise a substrate to which a gene or a cDNA or oligonucleotide corresponding to a fragment thereof is attached, and reagents, agents, and enzymes for constructing a fluorescently labeled probe. In addition, the substrate may comprise a control gene or a cDNA or oligonucleotide corresponding to a fragment thereof.

In addition, the diagnostic kit of the present invention may comprise essential elements necessary for performing ELISA. The ELISA kit may comprise an antibody specific to the protein. The antibody has high specificity and affinity for the marker protein, with no cross-reactivity to other proteins, and may be a monoclonal antibody, a polyclonal antibody, or a recombinant antibody. Furthermore, the ELISA kit may comprise an antibody specific to a control protein. In addition, the ELISA kit may further comprise reagents capable of detecting the bound antibody, for example, a labeled secondary antibody, chromophores, an enzyme (e.g., conjugated with the antibody) and a substrate thereof, or other substance capable of binding to the antibody.

In the diagnostic kit of the present invention, as a fixture for antigen-antibody binding reaction, there may be used a well plate synthesized from a nitrocellulose membrane, a PVDF membrane, a polyvinyl resin or a polystyrene resin, or a glass slide made of glass, without being limited thereto.

In the diagnostic kit of the present invention, a label for the secondary antibody is preferably a conventional chromogenic agent for color development, and examples of the label include, but are not limited to, fluoresceins such as HRP (horseradish peroxidase), alkaline phosphatase, colloid gold, FITC (poly L-lysine-fluorescein isothiocyanate), RITC (rhodamine-B-isothiocyanate), and dyes.

In the diagnostic kit of the present invention, a chromogenic substrate for inducing color development is preferably selected depending on the label for color development, and may be TMB (3,3′,5,5′-tetramethyl benzidine), ABTS [2,2′-azino-bis(3-ethylbenzothiazoline-6-sulfonic acid)], or OPD (o-phenylenediamine). At this time, the chromogenic substrate is more preferably provided as dissolved in buffer (0.1M NaOAc, pH 5.5). A chromogenic substrate such as TMB is degraded by HRP, used as a label for the secondary antibody conjugate, to form a chromogen, and the presence of the marker protein is detected by visually checking the degree of deposition of the chromogen.

The washing solution in the diagnostic kit of the present invention preferably comprises phosphate buffer, NaCl and Tween 20. More preferably, the washing solution is a buffer solution (PBST) consisting of 0.02 M phosphate buffer, 0.13 M NaCl, and 0.05% Tween 20. After the antigen-antibody binding reaction, the secondary antibody is allowed to react with the antigen-antibody complex, and then the resulting conjugate is washed 3 to 6 times with a suitable amount of the washing solution added to the fixture. As the reaction stop solution, a sulfuric acid solution (H2SO4) is preferably used.

According to yet another embodiment of the present invention, the present invention is directed to a method for providing information for cancer diagnosis, the method comprising a step of measuring the expression level of either a polypeptide represented by any one of SEQ ID NOs: 2 and 3, or a gene encoding the polypeptide, in a biological sample isolated from a subject of interest.

In the present invention, the “subject of interest” refers to a subject in whom whether or not the cancer has developed is uncertain and who has a high likelihood of developing the cancer.

In the present invention, the “biological sample” refers to any material, biological fluid, tissue or cells obtained or derived from the subject. For example, the biological sample may include whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid. Preferably, the biological sample may be a liquid biopsy (e.g., patient's tissue, cells, blood, serum, plasma, saliva, sputum or ascites, etc.) collected for histopathological examination by inserting a hollow needle or the like into an in vivo organ without incision of the skin of a patient having a high likelihood of developing cancer.

The method of the present invention may comprise a step of measuring the expression level of either the polypeptide represented by any one of SEQ ID NOs: 2 and 3, or a gene encoding the polypeptide, in a biological sample isolated as described above.

In the present invention, the step of measuring the expression level may be a step of measuring the expression level of erythroblastic oncogene B-2 (ERBB2) or a gene encoding the same.

In the present invention, the step of measuring the expression level is preferably a step of measuring both the expression level of the polypeptide represented by SEQ ID NO: 2 and the expression level of the polypeptide represented by SEQ ID NO: 3.

In the present invention, the step of measuring the expression level is preferably a step of measuring both the expression level of the gene encoding the polypeptide represented by SEQ ID NO: 2 and the expression level of the gene encoding the polypeptide represented by SEQ ID NO: 3.

In the present invention, an agent for measuring the expression level of the polypeptide represented by any one of SEQ ID NOs: 2 and 3 is not particularly limited, but preferably may comprise at least one selected from the group consisting of an antibody, an oligopeptide, a ligand, a peptide nucleic acid (PNA) and an aptamer, which bind specifically to the polypeptide.

In the present invention, methods for measurement or comparative analysis of the expression level of the polypeptide include, but are not limited to, protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoeletrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, and ELISA (enzyme-linked immunosorbent assay).

In the present invention, a method for measurement or comparative analysis of the expression level of the polypeptide represented by any one of SEQ ID NOs: 2 and 3 may be performed by a multiple-reaction monitoring (MRM) method.

In the present invention, the multiple-reaction monitoring method may be performed using mass-spectrometry, preferably triple-quadrupole mass spectrometry.

In the present invention, the multiple-reaction monitoring (MRM) method using mass-spectrometry is an analysis technique capable of monitoring a change in concentration of a specific analyte by selectively isolating, detecting and quantifying the specific analyte. MRM is a method that can quantitatively and accurately measure multiple substances such as trace amounts of biomarkers present in a biological sample. In MRM, mother ions among the ion fragments generated in an ionization source are selectively transmitted to a collision tube by a first mass filter Q1. Then, the mother ions arriving at the collision tube collide with an internal collision gas, are fragmented to generate daughter ions which are then sent to a second mass filter Q2, where only characteristic ions are transmitted to a detection unit. MRM is an analysis method with high selectivity and sensitivity that can detect only information on a component of interest. MRM is used for quantitative analysis of small molecules and is used to diagnose specific genetic diseases. The MRM method has advantages in that it is easy to simultaneously measure multiple peptides, and it is possible to confirm the relative concentration difference of protein diagnostic marker candidates between a normal person and a cancer patient without using an antibody. In addition, since the MRM analysis method has excellent sensitivity and selectivity, it has been introduced for the analysis of complex proteins and peptides in blood, particularly in proteomic analysis using a mass spectrometer (Anderson L. et al., Mol Cell Proteomics, 5: 375-88, 2006; DeSouza, L. V. et al., Anal. Chem., 81: 3462-70, 2009).

In the present invention, the expression level of the polypeptide represented by any one of SEQ ID NOs: 2 and 3 may be measured by the multiple-reaction monitoring method.

In the present invention, to analyze the expression level of the polypeptide represented by any one of SEQ ID NOs: 2 and 3 by the multiple-reaction monitoring method, a pair of mother and daughter ions in the selected target peptide may be selected. Table 1 below shows information about the pair of mother and daughter ions.

TABLE 1

| Gene | | | Target peptide | Mother ion | | |
|---|---|---|---|---|---|---|
| name | Protein name | uniprotKB | sequence | (m/z) | Daughter ion (m/z) | |
| ERBB2 | Erbb2 | P04626 | QVPLQR | 370.724 | 612.383 | +2y5 |
| | (erythroblastic | | (SEQ ID NO: 2) | | 513.314 | +2y4 |
| | oncogene B-2) | | | | 416.262 | +2y3 |
| | | | | | 303.178 | +2y2 |
| | | | | | 306.695 | +2y5+2 |
| | | | | | 257.161 | +2y4+2 |
| | | | | | 208.634 | +2y3+2 |
| | | | | | 152.092 | +2y2+2 |
| | | | SLTEILK | 402.247 | 603.371 | +2y5 |
| | | | (SEQ ID NO: 3) | | 502.324 | +2y4 |
| | | | | | 373.281 | +2y3 |
| | | | | | 260.197 | +2y2 |
| | | | | | 358.731 | +2y6+2 |
| | | | | | 302.189 | +2y5+2 |
| | | | | | 251.665 | +2y4+2 |
| | | | | | 187.144 | +2y3+2 |
| | | | | | 130.602 | +2y2+2 |

In the present invention, the polypeptide may be erythroblastic oncogene B-2 (ERBB2). The expression level of erythroblastic oncogene B-2 (ERBB2) protein in a biological sample isolated from a subject of interest may be measured using the polypeptide represented by any one of SEQ ID NOs: 2 and 3 as a target peptide.

In the present invention, it is preferable to use both the target peptide of the polypeptide represented by SEQ ID NO: 2 and the target peptide of the polypeptide represented by SEQ ID NO: 3.

In the present invention, in order to detect the erythroblastic oncogene B-2 (ERBB2) protein, a peptide is synthesized by substituting some amino acids of the target peptide of each of the proteins with a stable isotope. When the synthesized peptide is used as an internal standard substance in multiple-reaction monitoring analysis, the absolute amount of the protein in blood may also be measured, thus further increasing the accuracy of the analysis.

In the present invention, as the internal standard substance, any internal standard substance that is generally used in the multiple-reaction monitoring analysis may be used. For example, E. coli beta-galactosidase may be used. The target peptide representing the E. coli beta-galactosidase may consist of the polypeptide represented by SEQ ID NO: 8, and mother and daughter ions thereof may have mass-to-charge ratios of 542.3 m/z and 636.3 m/z, respectively, but are not limited thereto.

In addition, in the present invention, in order to measure the absolute amount of the erythroblastic oncogene B-2 (ERBB2) protein in blood, a specific peptide synthesized by substituting some amino acids of the target peptide with a stable isotope is used as an internal standard substance. In this case, the amino acids substituted with the isotope are preferably lysine or arginine, but are not limited thereto. Here, as the synthesized peptide, an isolated peptide with a purity of 95% or higher is preferably used.

Meanwhile, in the present invention, an agent for measuring the expression level of the gene encoding the polypeptide represented by any one of SEQ ID NOs: 2 and 3 may comprise at least one selected from the group consisting of a primer, a probe and an antisense nucleotide, which bind specifically to the gene.

In the present invention, to measure the presence and expression level of the gene encoding the polypeptide represented by any one of SEQ ID NOs: 2 and 3, an analysis method of measuring the mRNA level of the gene may be used. Examples of the analysis method include, but are not limited to, reverse transcription-polymerase chain reaction (RT-PCR), competitive RT-PCR, real-time RT-PCR, RNase protection assay (RPA), Northern blotting, and DNA chip assay.

In one embodiment of the present invention, if the measured expression level of either the polypeptide represented by any one of SEQ ID NOs: 2 and 3, or the gene encoding the polypeptide, in the biological sample isolated from the subject of interest, increases or decreases compared to a normal control, it may be predicted that the subject has a high likelihood of developing the cancer.

In other embodiments of the present invention, it is possible to predict responsiveness to therapy, preferably responsiveness to anticancer chemotherapy or immunotherapy, by measuring the expression level of either the polypeptide represented by any one of SEQ ID NOs: 2 and 3, or the gene encoding the polypeptide, in the biological sample isolated from the subject of interest.

In another embodiment of the present invention, it is possible to predict the prognosis of a subject of interest, preferably the prognosis after surgical operation, by measuring the expression level of either the polypeptide represented by any one of SEQ ID NOs: 2 and 3, or the gene encoding the polypeptide, in a biological sample isolated from the subject of interest. Here, the subject of interest may be a subject who has had cancer and has undergone surgical resection.

In another embodiment of the present invention, it is possible to predict the stage of cancer in a subject of interest by measuring the expression level of either the polypeptide represented by any one of SEQ ID NOs: 2 and 3, or the gene encoding the polypeptide, in a biological sample isolated from the subject of interest.

In the present invention, the “stage” refers to the extent to which cancer cells have spread or the stage of cancer progression. The international classification according to the status of cancer progression generally follows the TNM stage classification. Here, ‘T (Tumor Size)’ is a classification according to the size of the primary tumor, ‘N (Lymph Node)’ is a classification according to the degree of lymph node metastasis, and ‘M (Metastasis)’ is a classification according to whether cancer has metastasized to other organs. Detailed classification for T, N and M is shown in Table 2 below, and the stage classification of cancer according to T, N and M is shown in Table 3 below.

TABLE 2

| TNM stage | | Definition |
|---|---|---|
| Size of the | T0 | Tumor cells that show the appearance of a malignant |
| primary | | tumor, but are localized to the mucous membrane or |
| tumor | | epithelium of origin, and have not yet invaded the |
| (T stage) | | basement membrane. |
| | T1 | A lesion or tumor confined to the organ of origin, |
| | | which is mobile and has not invaded adjacent and |
| | | surrounding tissues. |
| | T2 | A tumor with a size of about 2 to 5 cm. |
| | T3 | A tumor having a size larger than T2 but localized |
| | | to the organ. |
| | T4 | Adhesion and infiltration into surrounding tissues. |
| Lymph | N0 | There is no evidence of lymph node involvement. |
| node status | N1 | Invades one palpable and mobile lymph node (1 to |
| (N stage) | | 2 cm or larger, usually up to 3 cm in size) limited |
| | | to the first station. |
| | N2 | Palpable, partially mobile and firm or hard lymph |
| | | nodes. There is microscopic evidence of invasion, |
| | | and involved nodes are clinically entangled and |
| | | show contralateral or bilateral involvement (3 to |
| | | 5 cm). |
| | N3 | Lymph nodes that are completely fixed, pass |
| | | through the capsule, are completely fixed to bones, |
| | | large blood vessels, skin, nerves, etc., and have a |
| | | size of 6 cm or more. |
| Distant | M0 | There is no distant metastasis. |
| metastasis | M1 | There is distant metastasis. |
| (M stage) | | |

TABLE 3

| | | | | |
|---|---|---|---|---|
| Stage | | | | |
| classification | T1 | T2 | T3 | T4 |

| | | |
|---|---|---|
| N0 | Stage 1 | Stage 2 |

| | |
|---|---|
| N1 | Stage 3 |
| N2 | |
| N3 | |
| M1 | Stage 4 |

In another embodiment of the present invention, it is possible to predict the likelihood of recurrence of cancer by measuring the expression level of either the polypeptide represented by any one of SEQ ID NOs: 2 and 3, or the gene encoding the polypeptide, in a biological sample isolated from a subject of interest.

In the present invention, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

According to another embodiment of the present invention, the present invention is directed to a method for screening a drug for preventing or treating cancer, the method comprising steps of:

(a) treating either a sample isolated from a cancer subject or a cancer disease animal model with a candidate drug; and (b) measuring the expression level of either the polypeptide represented by any one of SEQ ID NOs: 2 and 3, or the gene encoding the polypeptide, in the sample or cancer disease animal model treated with the candidate drug.

In the present invention, the “sample” includes tissue, cells, whole blood, serum, blood plasma, tissue autopsy sample (e.g., brain, skin, lymph node, spinal cord, etc.), cell culture supernatant, disrupted eukaryotic cells and bacterial expression system, but is not limited. With regard to the purposes of the present invention, the isolated sample is preferably cells or tissue isolated from a cancer subject, but is not limited thereto. The biological sample may be allowed to react with the candidate drug for preventing or treating cancer in a manipulated or unmanipulated state.

In the present invention, the term “cancer disease animal model” refers to an animal other than humans, which is an animal that may be determined by a person skilled in the art to be in a pathological state of cancer.

In the present invention, before the sample isolated from the cancer subject or the cancer disease animal mode is treated with the candidate drug, a step of measuring the expression level of either the polypeptide represented by any one of SEQ ID NOs: 2 and 3, or the gene encoding the polypeptide, may be performed.

The term “candidate drug” in the present invention refers to a substance that may be applied to a cancer patient to alleviate or beneficially change the patient's symptoms caused by cancer. The candidate drug is a substance capable of reducing the expression or activity of either the polypeptide represented by any one of SEQ ID NOs: 2 and 3, or the gene encoding the polypeptide. Examples of the candidate drug include, but are not limited to, low-molecular-weight compounds, antibodies, antisense nucleotides, small interfering RNAs, short hairpin RNAs, nucleic acids, proteins, peptides, other extracts or natural products.

In the present invention, the method of measuring the expression level of either the polypeptide represented by any one of SEQ ID NOs: 2 and 3, or the gene encoding the polypeptide, in the sample or the cancer disease animal model before or after treatment with the candidate drug, and the agent used in the method, overlap with those described above with respect to the method for providing information for cancer diagnosis, and thus detailed description thereof will be omitted herein.

In the present invention, the method may further comprise step (c) of determining that the candidate drug is the drug for preventing or treating cancer, when the expression level of either the polypeptide represented by any one of SEQ ID NOs: 2 and 3, or the gene encoding the polypeptide, measured in step (b), decreases or increases compared to that before treatment with the candidate drug.

Hereinafter, the present invention will be described in detail with reference to examples. However, the following examples serve merely to illustrate the present invention, and the scope of the present invention is not limited by the following examples.

### EXAMPLES

### Example 1

### Detection of Target Peptide for Measurement of Erythroblastic Oncogene B-2 (ERBB2)

1. Preparation of Samples

In order to evaluate the breast cancer diagnostic accuracy of a biomarker combination of the present invention, plasma was isolated from blood samples obtained from breast cancer patients and normal control persons, and total protein was quantified by Bradford assay. 200 μg of the total protein was modified with urea, and then reduced with dithiothreitol (DTT) and alkylated by iodoacetamide. Thereafter, trypsin was added thereto to form a peptide, and salt was removed using a C18 column. As an internal standard substance, a synthetic product in which the amino acid group attached to the end of the peptide is substituted with an isotope was used.

2. Performing Multiple-Reaction Monitoring Using Triple-Quadrupole Mass Spectrometry

For triple-quadrupole mass spectrometry, target peptides of erythroblastic oncogene B-2 (ERBB2) according to the present invention and pairs of mother and daughter ions thereof were selected, and the results are shown in Table 4 below.

TABLE 4

| Gene | | | Target peptide | Mother ion | | |
|---|---|---|---|---|---|---|
| name | Protein name | uniprotKB | sequence | (m/z) | Daughter ion (m/z) | |
| ERBB2 | Erbb2 | P04626 | QVPLQR | 370.724 | 612.383 | +2y5 |
| | (erythroblastic | | (SEQ ID NO: 2) | | 513.314 | +2y4 |
| | oncogene B-2) | | | | 416.262 | +2y3 |
| | | | | | 303.178 | +2y2 |
| | | | | | 306.695 | +2y5+2 |
| | | | | | 257.161 | +2y4+2 |
| | | | | | 208.634 | +2y3+2 |
| | | | | | 152.092 | +2y2+2 |
| | | | SLTEILK | 402.247 | 603.371 | +2y5 |
| | | | (SEQ ID NO: 3) | | 502.324 | +2y4 |
| | | | | | 373.281 | +2y3 |
| | | | | | 260.197 | +2y2 |
| | | | | | 358.731 | +2y6+2 |
| | | | | | 302.189 | +2y5+2 |
| | | | | | 251.665 | +2y4+2 |
| | | | | | 187.144 | +2y3+2 |
| | | | | | 130.602 | +2y2+2 |

The final samples prepared in Example 1.1 were subjected to reverse-phase resin chromatography to separate plasma peptide fragments, and MRM spectra of each peptide were obtained using triple quadrupole mass spectrometry (instrument: 5500 Qtrap, AB Sciex, USA). Here, the reverse-phase resin chromatography was performed with HALO™ C18 column (Eksigent, USA) using a 5% to 40% acetonitrile concentration gradient for 45 minutes. Quantitative information was confirmed by calculating the peak area of the MRM chromatogram of each target peptide by MultiQuant™ computer quantitative analysis program (AB Sciex, USA). Here, the quantitative value of each target peptide was expressed as a percentage relative to the peak area of the MRM chromatogram of E. coli beta-galactosidase added as an internal standard substance. The difference in the expression level of erythroblastic oncogene B-2 (ERBB2) between the breast cancer patients and the non-patient control patients was determined by calculating the MRM chromatogram area ratio of each peptide.

3. Evaluation of Breast Cancer Diagnostic Accuracy of Each Target Peptide

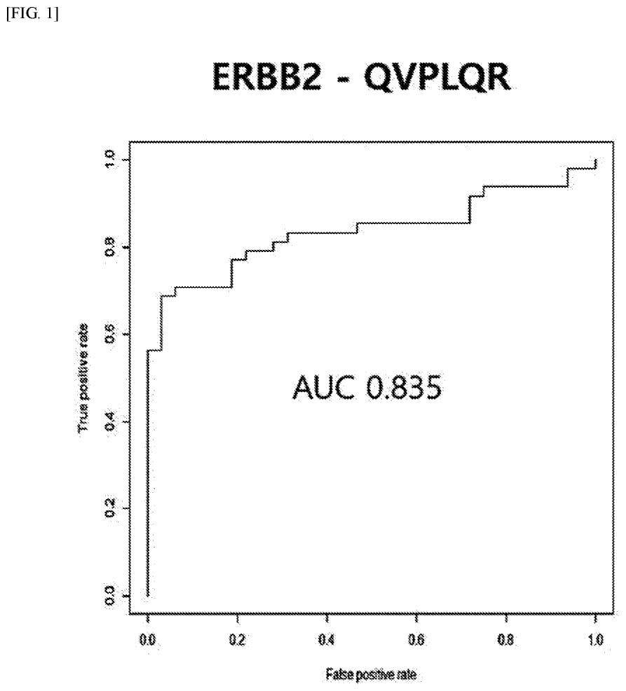

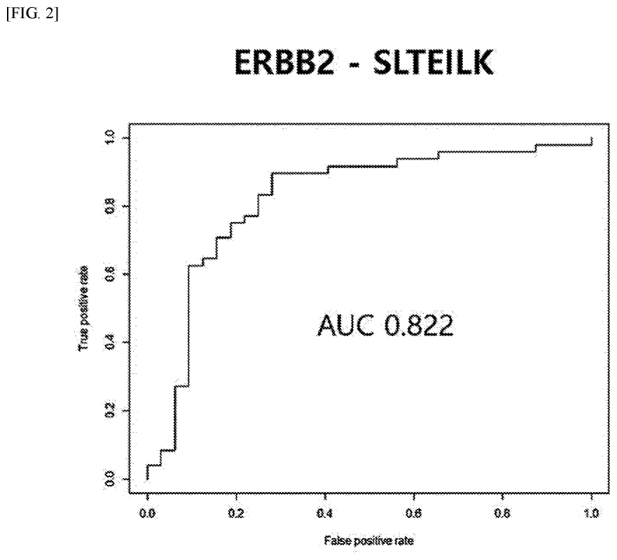

The breast cancer diagnostic efficiency of each of the markers of SEQ ID NOs: 2 and 3 for erythroblastic oncogene B-2 (ERBB2), identified in Example 1.2, was evaluated, and the results are shown in FIGS. 1 and 2 as receiver operating characteristic (ROC) graphs for the breast cancer patients and the non-patient control persons.

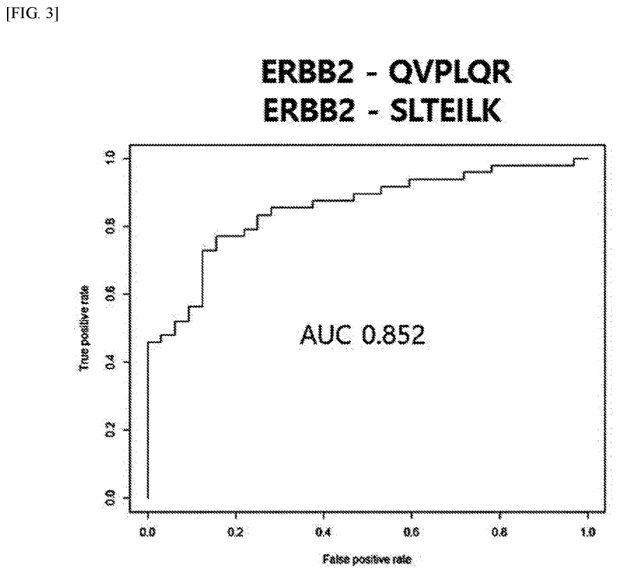

In addition, the quantitative results of each of the markers of SEQ ID NOs: 2 and 3, identified in Example 1.2, were unified through logistic regression, and a single diagnostic marker consisting of the polypeptides represented by SEQ ID NOs: 2 and 3 as target peptides was prepared and the breast cancer diagnostic efficiency thereof was evaluated. The results are shown in FIG. 3 as receiver operating characteristic (ROC) graphs for breast cancer patients and non-patient control persons.

As shown in FIGS. 1 to 3 showing receiver operating characteristic (ROC) graphs obtained for breast cancer patients and normal control persons, it could be seen that the use of the polypeptide represented by SEQ ID NO: 2 or 3 as a target peptide according to one embodiment of the present invention showed AUC (area under the curve) values of 0.835 and 0.822, respectively, indicating that the breast cancer diagnostic accuracy of each of the polypeptides was very high. In particular, as shown in FIG. 3, the use of a combination of the polypeptide represented by SEQ ID NO: 2 and the polypeptide represented by SEQ ID NO: 3 as a target peptide showed an AUC of 0.852, indicating that the combination of the polypeptides had significantly improved breast cancer diagnostic accuracy.

Accordingly, it could be seen that the erythroblastic oncogene B-2 (ERBB2) marker comprising any one of the target peptides represented by SEQ ID NOs: 2 and 3 could diagnose breast cancer with high accuracy.

In addition, it could be seen that the erythroblastic oncogene B-2 (ERBB2) marker comprising a combination of the target peptides represented by SEQ ID NOs: 2 and 3 could diagnose breast cancer with higher accuracy than each of the target peptides.

### Comparative Example 1

### Comparison of Breast Cancer Diagnostic Accuracy Between Target Peptides

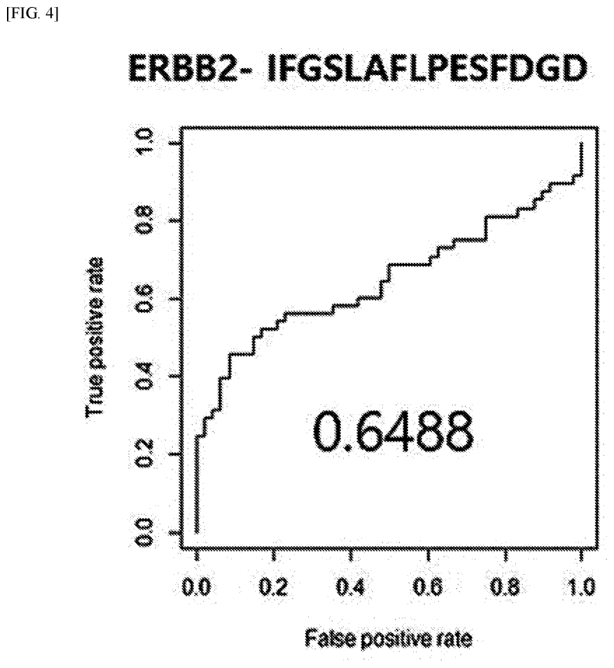

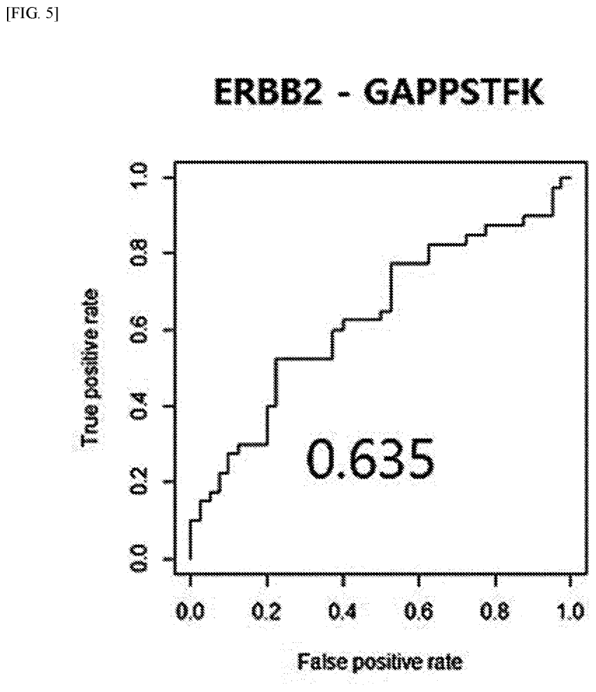

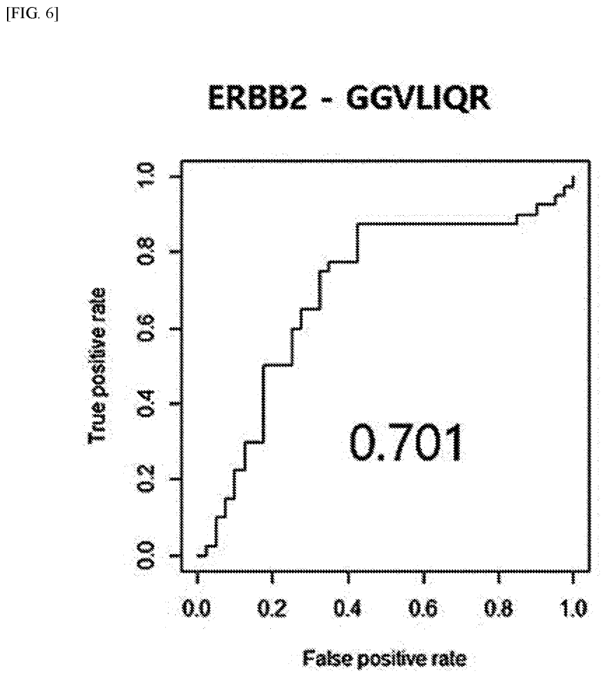

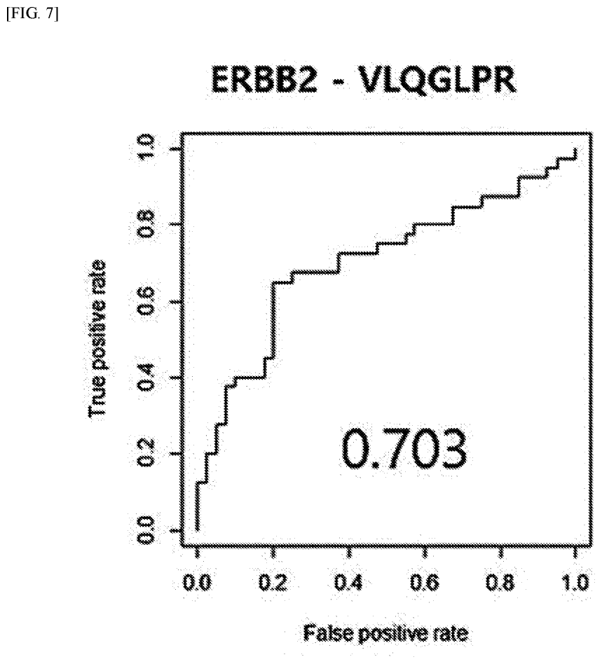

While the breast cancer diagnostic accuracy was measured in the same manner as in Example 1, target peptides of erythroblastic oncogene B-2 (ERBB2) and pairs of mother and daughter ions thereof were selected by performing multiple-reaction monitoring using triple-quadrupole mass spectrometry, and the results are shown in Table 5 below. The breast cancer diagnostic efficiency of each of the target peptides was evaluated, and the results are shown in FIGS. 4 to 7 as receiver operating characteristic (ROC) graphs for breast cancer patients and non-patient control persons.

TABLE 5

| Gene | | | Target peptide | Mother ion | | |
|---|---|---|---|---|---|---|
| name | Protein name | uniprotKB | sequence | (m/z) | Daughter ion (m/z) | |
| ERBB2 | Erbb2 | P04626 | IFGSLAFLPESFDGD | 661.983 | 388.183 | +3y4 |
| | (Erythroblastic | | (SEQ ID NO: 4) | | | |
| | oncogene B-2) | | GAPPSTFK | 402.716 | 676.367 | +2y6 |
| | | | (SEQ ID NO: 5) | | | |
| | | | GGVLIQR | 371.732 | 529.346 | +2y4 |
| | | | (SEQ ID NO: 6) | | | |
| | | | VLQGLPR | 391.748 | 570.336 | +2y5 |
| | | | (SEQ ID NO: 7) | | | |

As shown in FIGS. 4 to 7 showing receiver operating characteristic (ROC) graphs obtained for breast cancer patients and non-patient control persons using the polypeptide represented by any one of SEQ ID NOs: 4 to 7 as a target peptide according to the present invention, it could be seen that each of the polypeptides showed an AUC (area under the curve) value of 0.635 to 0.703, indicating that each of the polypeptides has a very low accuracy.

Accordingly, it can be seen that the use of any one of the polypeptides represented by SEQ ID NOs: 2 and 3 as a target peptide according to the present invention can diagnose breast cancer with high accuracy, and that the use of a combination of the target peptides can significantly increase the accuracy of diagnosis.

### INDUSTRIAL APPLICABILITY

The present invention relates to a composition capable of diagnosing cancer, a diagnostic kit comprising the same, and a method for providing information for diagnosing cancer using the composition.

### Sequence List Free Text

```
SEQ ID NO 1: Erythroblastic oncogene B-2 (ERBB2)
                10 20 30 40 50
MELAALCRWG LLLALLPPGA ASTQVCTGTD MKLRLPASPE THLDMLRHLY
                60 70 80 90 100
QGCQVVQGNL ELTYLPTNAS LSFLQDIQEV QGYVLIAHNQ VRQVPLQRLR
              110 120 130 140 150
IVRGTQLFED NYALAVLDNG DPLNNTTPVT GASPGGLREL QLRSLTEILK
              160 170 180 190 200
GGVLIQRNPQ LCYQDTILWK DIFHKNNQLA LTLIDTNRSR ACHPCSPMCK
              210 220 230 240 250
GSRCWGESSE DCQSLTRTVC AGGCARCKGP LPTDCCHEQC AAGCTGPKHS
              260 270 280 290 300
DCLACLHFNH SGICELHCPA LVTYNTDTFE SMPNPEGRYT FGASCVTACP
              310 320 330 340 350
YNYLSTDVGS CTLVCPLHNQ EVTAEDGTQR CEKCSKPCAR VCYGLGMEHL
              360 370 380 390 400
REVRAVTSAN IQEFAGCKKI FGSLAFLPES FDGDPASNTA PLQPEQLQVF
              410 420 430 440 450
ETLEEITGYL YISAWPDSLP DLSVFQNLQV IRGRILHNGA YSLTLQGLGI
              460 470 480 490 500
SWLGLRSLRE LGSGLALIHH NTHLCFVHTV PWDQLFRNPH QALLHTANRP
              510 520 530 540 550
EDECVGEGLA CHQLCARGHC WGPGPTQCVN CSQFLRGQEC VEECRVLQGL
              560 570 580 590 600
PREYVNARHC LPCHPECQPQ NGSVTCFGPE ADQCVACAHY KDPPFCVARC
              610 620 630 640 650
PSGVKPDLSY MPIWKFPDEE GACQPCPINC THSCVDLDDK GCPAEQRASP
              660 670 680 690 700
LTSIISAVVG ILLVVVLGVV FGILIKRRQQ KIRKYTMRRL LQETELVEPL
              710 720 730 740 750
TPSGAMPNQA QMRILKETEL RKVKVLGSGA FGTVYKGIWI PDGENVKIPV
              760 770 780 790 800
AIKVLRENTS PKANKEILDE AYVMAGVGSP YVSRLLGICL TSTVQLVTQL
              810 820 830 840 850
MPYGCLLDHV RENRGRLGSQ DLLNWCMQIA KGMSYLEDVR LVHRDLAARN
              860 870 880 890 900
VLVKSPNHVK ITDFGLARLL DIDETEYHAD GGKVPIKWMA LESILRRRFT
              910 920 930 940 950
HQSDVWSYGV TVWELMTFGA KPYDGIPARE IPDLLEKGER LPQPPICTID
              960 970 980 990 1000
VYMIMVKCWM IDSECRPRFR ELVSEFSRMA RDPQRFVVIQ NEDLGPASPL
            1010 1020 1030 1040 1050
DSTFYRSLLE DDDMGDLVDA EEYLVPQQGF FCPDPAPGAG GMVHHRHRSS
            1060 1070 1080 1090 1100
STRSGGGDLT LGLEPSEEEA PRSPLAPSEG AGSDVFDGDL GMGAAKGLQS
            1110 1120 1130 1140 1150
LPTHDPSPLQ RYSEDPTVPL PSETDGYVAP LTCSPQPEYV NQPDVRPQPP
            1160 1170 1180 1190 1200
SPREGPLPAA RPAGATLERP KTLSPGKNGV VKDVFAFGGA VENPEYLTPQ
            1210 1220 1230 1240 1250
GGAAPQPHPP PAFSPAFDNL YYWDQDPPER GAPPSTFKGT PTAENPEYLG
```

LDVPV

SEQ ID NO: 2: Target peptide of erythroblastic oncogene B-2 (ERBB2)

QVPLQR

SEQ ID NO: 3: Target peptide of erythroblastic oncogene B-2 (ERBB2)

SLTEILK

SEQ ID NO: 4: Target peptide of erythroblastic oncogene B-2 (ERBB2)

IFGSLAFLPESFDGD

SEQ ID NO: 5: Target peptide of erythroblastic oncogene B-2 (ERBB2)

GAPPSTFK

SEQ ID NO: 6: Target peptide of erythroblastic oncogene B-2 (ERBB2)

GGVLIQR

SEQ ID NO: 7: Target peptide of erythroblastic oncogene B-2 (ERBB2)

VLQGLPR

SEQ ID NO: 8: Target peptide of E. coli beta-galactosidase

GDFQFNISR

1. A cancer diagnostic marker comprising a polypeptide represented by any one of SEQ ID NOs: 2 and 3.

2. (canceled)

3. A cancer diagnostic composition containing an agent for measuring an expression level of either the polypeptide represented by any one of SEQ ID NOs:
2 and 3 of claim 1, or a gene encoding the polypeptide.

4. (canceled)

5. The cancer diagnostic composition of claim 3, wherein the agent for measuring the expression level of the polypeptide represented by any one of SEQ ID NOs: 2 and 3 comprises at least one selected from the group consisting of an antibody, an oligopeptide, a ligand, a peptide nucleic acid (PNA) and an aptamer, which bind specifically to the polypeptide.

6. The cancer diagnostic composition of claim 3, wherein the agent for measuring the expression level of the gene encoding the polypeptide represented by any one of SEQ ID NOs: 2 and 3 comprises at least one selected from the group consisting of a primer, a probe and an antisense nucleotide, which bind specifically to the gene encoding the polypeptide.

7-8. (canceled)

9. A method for providing information for cancer diagnosis, the method comprising a step of measuring an expression level of either a polypeptide represented by any one of SEQ ID NOs: 2 and 3, or a gene encoding the polypeptide, in a biological sample isolated from a subject of interest.

10. The method of claim 9, wherein the biological sample is whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

11. The method of claim 9, wherein the agent for measuring the expression level of the polypeptide represented by any one of SEQ ID NOs: 2 and 3 comprises at least one selected from the group consisting of an antibody, an oligopeptide, a ligand, a peptide nucleic acid (PNA) and an aptamer, which bind specifically to the polypeptide represented by any one of SEQ ID NOs: 2 and 3.

12. The method of claim 9, wherein the measuring of the expression level of the polypeptide represented by any one of SEQ ID NOs: 2 and 3 is performed by protein chip assay, immunoassay, ligand binding assay, matrix assisted laser desorption/ionization time of flight mass spectrometry (MALDI-TOF) assay, surface enhanced laser desorption/ionization time of flight mass spectrometry (SELDI-TOF) assay, radioimmunoassay, radioimmunodiffusion, Ouchterlony immunodiffusion, rocket immunoeletrophoresis, immunohistochemical staining, complement fixation assay, two-dimensional electrophoresis assay, liquid chromatography-mass spectrometry (LC-MS), liquid chromatography-mass spectrometry/mass spectrometry (LC-MS/MS), Western blotting, or enzyme-linked immunosorbent assay (ELISA).

13. The method of claim 9, wherein the measuring of the expression level of the polypeptide represented by any one of SEQ ID NOs: 2 and 3 is performed by a multiple-reaction monitoring (MRM) method.

14. The method of claim 13, wherein a pair of mother and daughter ions of the polypeptide represented by SEQ ID NO: 2 as a target peptide may have mass-to-charge ratios of 370.724 m/z and 612.383, 513.314, 416.262, 303.178, 306.695, 257.161, 208.634 and 152.092 m/z, respectively, and a pair of mother and daughter ions of the polypeptide represented by SEQ ID NO: 3 as a target peptide may have mass-to-charge ratios of 402.247 m/z and 603.371, 502.324, 373.281, 260.197, 358.731, 302.189, 251.665, 187.144 and 130.602 m/z, respectively.

15. The method of claim 13, wherein an internal standard substance that is used in the multiple-reaction monitoring method is either a synthetic peptide obtained by substituting certain amino acids of the target peptide with an isotope, or E. coli beta-galactosidase.

16. The method of claim 15, wherein a target peptide representing the E. coli beta-galactosidase consists of a polypeptide represented by SEQ ID NO: 8, and mother and daughter ions thereof have mass-to-charge ratios of 542.3 m/z and 636.3 m/z, respectively.

17. The method of claim 9, wherein the agent for measuring the expression level of the gene encoding the polypeptide represented by any one of SEQ ID NOs: 2 and 3 comprises at least one selected from the group consisting of a primer, a probe and an antisense nucleotide, which bind specifically to the gene encoding the polypeptide.

18. The method of claim 9, wherein the measuring of the expression level of the gene encoding the polypeptide represented by any one of SEQ ID NOs: 2 and 3 is performed by reverse transcription-polymerase chain reaction (RT-PCR), competitive RT-PCR, real-time RT-PCR, RNase protection assay (RPA), Northern blotting, or DNA chip assay.

19. The method of claim 9, wherein, when the measured expression level of either the polypeptide represented by any one of SEQ ID NOs: 2 and 3, or the gene encoding the polypeptide, in the biological sample isolated from the subject of interest, increases compared to a normal control, it is predicted that the subject has a high likelihood of developing the cancer.

20-23. (canceled)

24. The method of claim 9, wherein the cancer is breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

25. A method for screening a drug for preventing or treating cancer, the method comprising steps of:
(a) treating either a sample isolated from a cancer subject or a cancer disease animal model with a candidate drug; and
(b) measuring an expression level of either a polypeptide represented by any one of SEQ ID NOs: 2 and 3, or a gene encoding the polypeptide, in the sample or cancer disease animal model treated with the candidate drug.

26. The method of claim 25, wherein the sample is cells or tissue isolated from the cancer subject.

27. The method of claim 25, further comprising step (c) of determining that the candidate drug is the drug for preventing or treating cancer, when the expression level of the polypeptide represented by any one of SEQ ID NOs: 2 and 3, or the gene encoding the polypeptide, measured in step (b), decreases or increases compared to that before treatment with the candidate drug.

28. The method of claim 25, wherein the cancer is breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.


SEQUENCE LISTING (8 sequences)

SEQ ID NO: 1 - protein, 1255 aa, Homo sapiens [sequence omitted]

SEQ ID NO: 2 - protein, 6 aa, Artificial Sequence
  ERBB2 protein fragment
QVPLQR

SEQ ID NO: 3 - protein, 7 aa, Artificial Sequence
  ERBB2 protein fragment
SLTEILK

SEQ ID NO: 4 - protein, 15 aa, Artificial Sequence
  ERBB2 protein fragment
IFGSLAFLPESFDGD

SEQ ID NO: 5 - protein, 8 aa, Artificial Sequence
  ERBB2 protein fragment
GAPPSTFK

SEQ ID NO: 6 - protein, 7 aa, Artificial Sequence
  ERBB2 protein fragment
GGVLIQR

SEQ ID NO: 7 - protein, 7 aa, Artificial Sequence
  ERBB2 protein fragment
VLQGLPR

SEQ ID NO: 8 - protein, 9 aa, Escherichia coli
GDFQFNISR